(19) United States
(12) Patent Application Publication
(10) Pub. No.: US 2024/0306939 A1
(43) Pub. Date: Sep. 19, 2024

(54) METHOD FOR MONITORING AND ANALYZING THE CARDIAC CONDITION OF AN INDIVIDUAL

(71) Applicant: WITHINGS, ISSY LES MOULINEAUX (FR)

(72) Inventors: Pierre BARTET, ISSY LES MOULINEAUX (FR); Nicolas GENAIN, ISSY LES MOULINEAUX (FR)

(21) Appl. No.: 18/257,733

(22) PCT Filed: Jan. 7, 2022
(86) PCT No.: PCT/FR2022/050045

§ 371 Date: Jun. 15, 2023

(30) Foreign Application Priority Data
Jan. 7, 2021 (FR) FR2100114

(51) Int. Cl. A61B 5/11; A61B 5/00; A61B 5/0205; A61B 5/08; A61B 5/361

(52) U.S. Cl. CPC A61B 5/1102; A61B 5/002; A61B 5/0205; A61B 5/361; A61B 5/6891; A61B 5/6892; A61B 5/7221; A61B 5/7246; A61B 5/7267; A61B 5/08; A61B 2560/045; A61B 2562/0204; A61B 2562/0247; A61B 2562/164

(57) ABSTRACT

A method of determining a characteristic of a cardiac condition of an individual, the method including a) measuring at least one signal representative of a cardiac activity of the individual repeatedly; c) processing at least part of the at least one measured signal; d) analyzing a time evolution of at least part of the at least one processed signal; e) providing a characteristic of the signal representative of the cardiac activity, based on procedures a) to d).

### TECHNICAL AREA

The present disclosure relates to methods for monitoring an individual's cardiac status during sleep.

### PREVIOUS TECHNIQUE

Early detection of a disease can be crucial to its treatment, and to taking full advantage of the possibilities and means of recovery.

As for heart-related illnesses, early detection without regular medical check-ups is often difficult, as some warning signs of heart-related problems such as fatigue or shortness of breath can have many origins.

Monitoring a person's sleep can provide information about heart problems of which the person concerned is often unaware.

To this end, various monitoring devices and processes have been proposed, configured to detect breathing, heartbeat or pressure signals.

An example of a method for detecting cardiac activity is illustrated by JP2013500757 A. An example of a method for calculating heart rate is illustrated by EP3456256.

However, these signals may have a low amplitude and be drowned in background noise, and it can be difficult to extract useful information from them.

### SUMMARY

This disclosure improves the situation.

A method of determining a cardiac condition of an individual is proposed for providing a characteristic of a cardiac signal (in particular a temporal characteristic relating to the regularity of the cardiac signal), said method comprising the following steps:
/a/ obtaining (e.g. by measuring) at least one signal representative of a cardiac activity of the individual repeatedly;
/c/ processing at least a part of the at least one obtained signal;
/d/ analyzing a temporal evolution of at least a part of the at least one processed signal;
/e/ provide a characteristic of the signal representative of cardiac activity, based on steps /a/ to /d/.

Identifying a characteristic enables early measures to be taken to prevent the onset of a disease, or, in the event that an onset of disease cannot be prevented, to provide suitable treatment for the individual in good time. This is particularly true when the characteristic of the cardiac signal is its irregularity.

In one embodiment, the method comprises the following step: /b/ determining a relevance of at least part of the at least one signal obtained, in order to determine whether the signal obtained is usable for determining a characteristic of the cardiac signal. Step /b/ can be performed at any time.

In one embodiment, step /b/ comprises an automatic learning step, implemented by an artificial intelligence.

An automatic learning step implements the principle of learning from experience, i.e. determining, on the basis of previously obtained signals, whether a signal or part of a signal is relevant and should be taken into account for determining an individual's cardiac state.

In one embodiment, the at least one signal representative of cardiac activity is a pressure signal and step /c/ comprises determining a ballistocardiogram.

Ballistocardiography is a technique for exploring the minute body movements caused by cardiac contraction. Here, in particular, a ballistocardiogram focuses attention and analysis on the minute body movements caused by cardiac contraction. Features of the cardiac state can be found in the ballistocardiogram, and analyzed when step /d/ is carried out. It can be noted that a ballistocardiogram can be obtained completely non-invasively, even without the individual's knowledge. We also note that a ballistocardiogram can be obtained without direct contact with the individual.

In one embodiment, step /c/ comprises calculating a self-similarity.

Self-similarity makes it possible to analyze and classify at least part of the at least one signal obtained, without the use of an external reference signal.

Self-similarity is concerned with all or some of the extrema of the signal obtained, and a score representing the sum of the distances between each extremum of the signal obtained and the nearest extremum of its time-shifted copy is calculated as a function of a time shift between the signal obtained and its time-shifted copy (hereinafter also called the reference signal).

Since the computational resources required to calculate a self-similarity are less than those required to implement similar methods such as autocorrelation, computational costs can be minimized.

In one embodiment, step /c/ comprises: calculating, for a portion of the representative signal, a set of similarity scores, said set of similarity scores associating similarity score values with respective time shifts. This set of similarity scores can take the form of a score function, whose variable is the time offset. For each portion of the representative signal, similarity scores are assigned to the different time shifts. In particular, the set of similarity scores can be calculated by comparing the portion with a plurality of recopies of the portion to which a respective time offset has been applied. A similarity score value is then assigned for each time shift, thereby generating the score set. This recopy shift method is an implementation of self-similarity. However, similarity scores can also be calculated using other methods.

In one embodiment, step /c/ comprises: stacking a plurality of sets of similarity scores calculated for a plurality of respective portions, to form a stack of representative signal self-similarity scores. The stack thus associates values of similarity scores of the representative signal with time shifts and portions of the representative signal. In this way, data on the similarity of the representative signal is obtained over time. This stack is then analyzed in step /d/ (in particular, as input to a processing algorithm).

In one embodiment, step /d/ is implemented at least in part by artificial intelligence.

The analysis of the temporal evolution of at least part of at least one signal processed by an artificial intelligence allows great flexibility in the implementation of step /d/. In this way, the process can be successful and provide a feature for a large number of signal shapes and patterns, thanks to the ability of the signal classification artificial intelligence to analyze.

In one embodiment, the artificial intelligence comprises an artificial neural network.

An artificial neural network implements the principle of learning from experience, enabling the results obtained in the analysis according to step /d/ to be optimized on the basis of previously obtained results.

In one embodiment, the artificial neural network comprises a two-dimensional convolutional neural network, i.e. a neural network with at least one two-dimensional convolution layer.

This type of neural network is specially adapted for image analysis, in particular for the detection and analysis of certain patterns and shapes in images. In practice, an image is constructed from a juxtaposition of several self-similarity results, and the image in question forms the input to the neural network. What's more, this type of network uses reduced memory and computing power, enabling it to be embedded in a device of reduced size and inexpensive design.

In one embodiment, at least some of steps /c/ to /e/ are carried out repeatedly, and a feature is provided every 1 minute to 5 minutes.

As a general rule, we monitor signals over a medium to long term in relation to the cardiac period.

In this way, a precise study of the temporal evolution of the identified characteristic can be carried out.

In one embodiment, step /b/ comprises:
analyzing the at least one signal obtained representative of a cardiac activity of the individual; and/or
measuring and analyzing at least one additional signal representative of a movement performed by the individual, a breath taken by the individual or a pressure exerted by the individual.

This step checks whether the conditions required to provide a feature are met, for example whether the individual is present and correctly positioned.

In one embodiment, the characterization of step /e/ comprises a heart rhythm irregularity or atrial fibrillation. Alternatively, the characterization of step /e/ may comprise an absence of irregularity. More generally, the characteristic of the representative signal may relate to the regularity or irregularity of the signal over time.

The process makes it possible to provide a characterization that is relevant and easy to understand for an individual with no medical training, such as “No particularities detected” or “Atrial fibrillation detected: Please contact your doctor”.

In one embodiment, at least part of the process is implemented while the individual is sleeping.

In this way, signals can be measured continuously and in the individual's state of rest, while minimizing disruptions to the measurements, i.e. avoiding, for example, tension or stress that the individual might experience during an examination in a doctor's surgery.

We can benefit from collecting signals over a long period of several hours of sleep a night, and repeat this acquisition every night, thus accumulating a large amount of data.

Another aspect of the disclosure comprises a processing unit (so-called first processing unit) for determining a characteristic of a cardiac state of an individual. The processing unit is configured to implement a method for determining a characteristic of a cardiac state of an individual as previously described. The processing unit may comprise a memory storing instructions corresponding to the previously described processes and a control circuit capable of executing said instructions.

In particular, the unit can implement the following steps:
/a/ obtaining (201) at least one signal representative of cardiac activity of the individual repeatedly;
/c/ processing (203) at least part of the at least one signal obtained;
/d/ analyzing (204) a temporal evolution of at least part of the at least one signal processed;
/e/ provide (205) a characteristic of the signal representative of cardiac activity based on steps /a/ to /d/.

Another aspect of the disclosure comprises a device for determining a characteristic of a cardiac state of an individual, the device comprising at least one sensor configured to measure at least one signal representative of a cardiac activity of the individual repeatedly, and the processing unit configured to implement the method of determining a characteristic, the device being configured to determine the cardiac state of the individual without being in physical contact with the individual. By “without being in physical contact with the individual” is meant without being in direct physical contact with the individual's body. In other words, there may be an object (e.g. a mattress and/or clothing) between the device and the individual's body.

This device implements the method for determining a characteristic of an individual's cardiac state.

The identification of a characteristic enables early measures to be taken to prevent the onset of a disease, or, in the event that an onset of disease cannot be prevented, to provide appropriate treatment for the individual in good time.

The individual wears no electrodes or other sensors on his or her body during the process. Totally non-invasive, the device is transparent to use for an individual, and even usable in certain pathological cases without the individual's knowledge. Useful signals can be obtained without direct contact with the individual.

In one embodiment, the device comprises a second processing unit remote from the device and configured to receive from the first processing unit, after implementation of the process by the first processing unit, data representative of at least a portion of the at least one measured signal and/or the at least one processed signal and/or the characteristic.

In this way, at least some of the measured and/or processed data and/or any other data relating to the feature can be transmitted to the second processing unit, for storage and/or visualization and/or further use.

In one embodiment, the second processing unit comprises a smartphone or tablet.

A “smartphone” or tablet can be used to save data, for example on the “smartphone” or tablet, a cloud or any other storage unit, to view data or to share data with a qualified person such as a doctor.

Another aspect of the disclosure comprises a computer program product having instructions for implementing the method of the present invention, when the computer program product is executed by a processor.

This program can use any programming language (object-oriented or otherwise), and be in the form of interpretable source code, partially compiled code or fully compiled code.

FIG. 7, described in detail below, may form the first processing unit configured to implement the method for determining a characteristic of an individual's cardiac condition.

### BRIEF DESCRIPTION OF THE DRAWINGS

Further features, details and advantages will become apparent from the detailed description below, and from an analysis of the appended drawings, on which:

FIG. 1 shows a device for implementing a method for determining a characteristic of an individual's cardiac condition.

FIG. 2 shows a flowchart of a process for determining a characteristic of a cardiac condition.

FIG. 3 shows the result of a two-dimensional self-similarity calculation based on a measured signal representative of an individual's cardiac activity, indicating sinus rhythm or atrial fibrillation respectively.

FIG. 4 shows the result of a three-dimensional self-similarity calculation based on a measured signal representative of an individual's cardiac activity, indicating sinus rhythm.

FIG. 5 shows the result of a three-dimensional self-similarity calculation based on a measured signal representative of an individual's cardiac activity, indicating atrial fibrillation.

FIG. 6 shows an illustration of an artificial neural network for implementing part of the process of determining a characteristic of an individual's cardiac state.

FIG. 7 is a schematic view of a processing unit configured to implement the process of determining a characteristic of an individual's cardiac condition.

### DESCRIPTION OF EMBODIMENTS

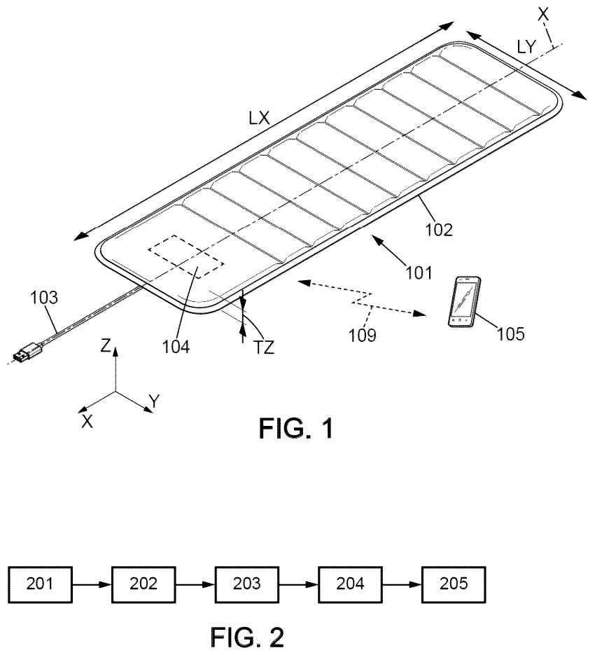

FIG. 1 describes a device 101 for determining a characteristic of an individual's cardiac condition.

The device 101 can be installed in a bed, for example under a mattress, to monitor the individual on the mattress during sleep, and identify heart problems such as atrial fibrillation.

The device 101 may comprise a strip of fabric 102 forming a sheath.

The device 101 may comprise at least one sensor 107, 108 configured to measure at least one signal representative of cardiac activity, such as a pressure sensor 107 or an acoustic sensor 108.

The fabric strip 102 may contain a pneumatic chamber acting as a pressure sensor 107.

In another variant, the pressure sensor 107 may comprise at least one piezoelectric sensor, enclosed by the fabric strip 102.

For the case where the pressure sensor 107 comprises a pneumatic chamber, and when the device 101 is arranged in a bed, the weight and movements of the individual present on the bed act on the pneumatic chamber, resulting in pressure variations.

The pressure signal can be, for example, a micro-vibration of the individual's body, generated by the heartbeat.

In addition, the fabric strip 102 can enclose a housing 104.

The housing 104 may include electronic means connected to a connection cable 103 comprising a wire and a USB connector, to provide electrical power to the device 101. Alternatively, the device may comprise a plug for connection to an electrical outlet. The device may also include a rechargeable battery.

The housing 104 can include a pressure converter, configured to transform a pressure signal, detected using the 107 pressure sensor, into a first voltage.

In addition, the housing 104 can include a microphone, acting as an acoustic sensor 108.

The acoustic sensor 108 can measure environmental sounds, such as breathing or snoring, and convert them into a second voltage.

The acoustic sensor 108 and the pressure converter can be electronically connected to a first processing unit 106 located in the housing 104.

The first processing unit 106 can be configured to implement all or part of the process for determining a characteristic of an individual's cardiac condition. This process is described in detail in FIG. 2.

The first and/or second voltage can be used to determine heart rate, heart rate amplitude and other parameters.

In one embodiment, the device comprises a display. This screen can be configured to display feature-related data such as “No features detected” or “Atrial fibrillation detected: Please contact your doctor”.

The first processing unit can be further configured to communicate, as shown by the dotted arrow 109 in FIG. 1, with a second processing unit.

The second processing unit can be configured to receive, by the first processing unit, at least part of the measured (representing the at least one measured signal) and/or processed data and/or any other data relating to the feature, for storage and/or visualization and/or any other subsequent use.

The second processing unit can be remote from the device, i.e. not physically linked to the device.

The second processing unit may be or comprise a smartphone 105 or tablet, enabling data to be saved, for example on the smartphone or tablet or in a cloud or other external device, data to be viewed or data to be shared with a qualified person such as a doctor.

A suitable application can be installed on the smartphone/tablet.

Data may be shared with a doctor at the customer's initiative, or automatically, in particular if a characteristic relating to an irregular heartbeat is identified by the device.

If the pressure sensor 107 comprises a pneumatic chamber, the pneumatic chamber can be inflated prior to use.

After use, the device 101 can be either stored, rolled up or folded so that it is compact when not in use.

In one embodiment, the device 101 can be rectangular in shape, with a length LX of between 50 mm and 800 mm, a width LY of between 10 mm and 400 mm, and a thickness TZ of less than 20 mm.

Due to the low thickness TZ of the device 101, the individual sleeping on the mattress is not disturbed by the device 101 when the device is installed under the mattress.

The shape of the pneumatic chamber can be substantially the same as the shape of the fabric strip 102.

FIG. 2 shows the flowchart of a process for determining a characteristic of an individual's cardiac condition. This process can be implemented by the device 101 shown in FIG. 1, which may comprise a first processing unit 106.

At least part of the process can be carried out while the individual is asleep.

The individual can be a human being, and the device 101 can be installed in a bed, for example under the mattress, in order to monitor the human being during sleep.

In another embodiment, the individual can be an animal, such as a dog, and the process can be carried out while the dog is sleeping in a basket.

In a first step 201, at least one signal representative of the individual's cardiac activity can be measured. This measurement can be carried out repeatedly, for example continuously over a period of time ranging from several minutes to several hours, or even a day.

This signal can be a pressure signal and/or an acoustic signal, detected by at least one pressure sensor 107 and/or acoustic sensor 108 included in the device 101.

The acoustic signal can be representative of the individual's breathing or snoring.

The pressure signal can be representative of minute body movements caused by a cardiac contraction made by the individual, but also of other movements when the individual moves in his sleep.

More generally, step 201 may comprise obtaining a signal representative of the cardiac activity being measured; in particular, the device that acquires the signal may be different from the device that implements steps 202, 203, 204, 205 of the method of the description.

In a second step 202, the measured signal can be used to determine the relevance of at least part of the measured signal.

Step 202 can include an automatic learning step, implemented by an artificial intelligence such as an artificial neural network.

To implement step 202, at least one measured signal representative of the individual's cardiac activity can be analyzed.

In another embodiment, at least one additional signal representative of movement by the individual, breathing by the individual or pressure exerted by the individual can be measured and analyzed.

In one variant, the evolution of the amplitude of a pressure signal can be observed, in particular the evolution from one pressure signal maximum to another. For example, a macroscopic movement performed by the individual may saturate the signal reading, i.e. if the individual moves during sleep, the signal measured by the pressure sensor configured to detect minute pressure signals may saturate. In this case, the result of step 202 for this part of the signal may be an error message, and a signal representative of cardiac activity cannot be extracted. No characteristic of the signal representative of cardiac activity can be provided in this case.

If no pressure or cardiac activity is detected, a result of step 202 may be that the individual is not on the bed or is not positioned correctly, and the feature cannot be provided accordingly.

In a third step 203, at least part of the measured signal is processed.

For the case where the signal representative of cardiac activity is a pressure signal, the third step 203 may involve determining a ballistocardiogram. A ballistocardiogram is a written record of minute body movements caused by a cardiac contraction. Characteristics of the cardiac state can be encoded in a ballistocardiogram.

The third step 203 can include signal similarity calculations, which can provide information about the rhythm and regularity of the cardiac signal.

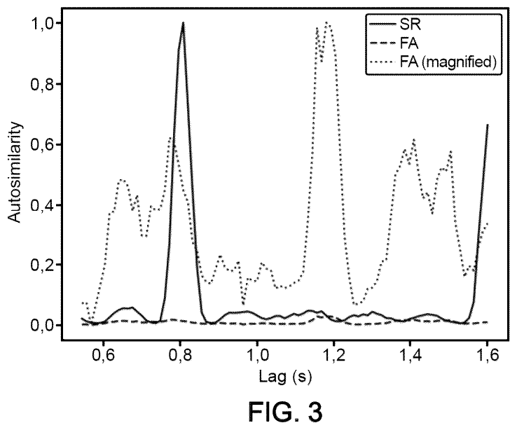

For example, in one embodiment, the third step 203 comprises a self-similarity calculation. The implementation and results of a self-similarity calculation are described in detail in FIGS. 3 to 5.

For example, in another embodiment, the third step may involve calculating an autocorrelation.

In a fourth step 204, a time trend of at least part of the at least one processed signal is analyzed. In particular, similarity score calculations can be used as inputs to a processing algorithm.

In one embodiment, the fourth step 204 is implemented at least in part by an artificial intelligence, allowing great flexibility in the implementation of the process. In this way, the process can succeed and provide a feature for different shapes or patterns in the signals/data, thanks to the ability of the artificial intelligence to classify the data to be analyzed.

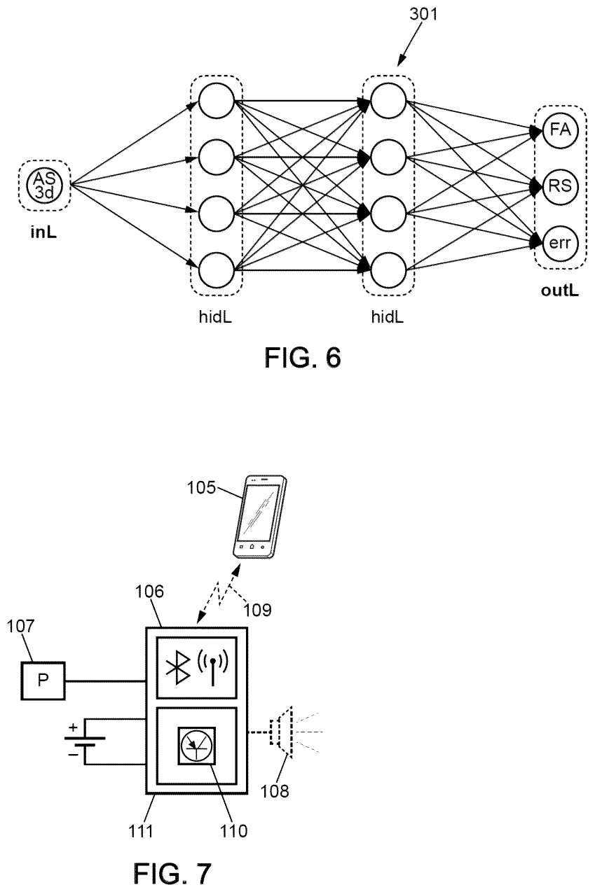

In one embodiment, the artificial intelligence comprises an artificial neural network such as a two-dimensional convolutional neural network 301 that comprises at least one two-dimensional convolutional hidden layer, as explained in the description relating to FIG. 6.

A two-dimensional convolutional neural network is especially suitable for image analysis, especially for detecting and analyzing certain patterns in images.

An artificial neural network is an algorithm that enables a processing unit 106 running this algorithm to learn from new data. An artificial neural network can learn by feeding the algorithm with data comprising processed recordings labeled with pressure variation and/or sound.

Labeled recordings can be classified, for example, according to the individual's gender, weight, age and type of cardiac signal characteristic.

In this way, a reference database can be created and enhanced over time. For each individual using the device according to FIG. 1, a qualified person such as a doctor can label the sound and pressure variation recordings in association with the profile.

The implementation of step 204 is described in detail in FIG. 6.

In a fifth step 205, a feature based on the first 201 to fourth 204 steps can be provided. The characteristic may relate to a temporal regularity or temporal irregularity of the signal.

For example, the fifth step 205 may include identification of atrial fibrillation or identification of an absence of abnormal cardiac activity.

It is not necessary for the process to be implemented in the order described above. In particular, the second step 202 can be implemented at any time during the process, for example directly after signal measurement, or at the same time as the third or fourth step. However, a feature such as “No peculiarities detected” or “Atrial fibrillation detected: Please contact your doctor”, is only provided if it is determined in the second step 202 that the measured signal is relevant. Otherwise, the process may provide an error message. Thus, the process notifies the user of the characteristic of the measured signal only if the measured signal is usable.

Furthermore, it is not necessary for the first step 201 to be completed to trigger implementation of the second step 202. The second 202, third 203 or fourth 204 steps can be implemented at least in part during signal measurement in the first step 201.

In one embodiment, part of the process, in particular any of steps 202, 203, 204 and 205, can be implemented by a second processing unit remote from the device, such as a smartphone.

In one embodiment, at least some of steps /c/ to /e/ are carried out repeatedly, and a feature is provided every 1 minute to 5 minutes.

FIG. 3 shows the two-dimensional result of a self-similarity calculation.

The self-similarity calculation can be carried out during the third step 203 of the process described in FIG. 2, based on the signal measured during the first step 201.

In an optional preliminary sub-step, a bandpass filter can be applied to the at least one measured signal to eliminate high-frequency background noise. In addition, the bandpass filter can be configured to eliminate low-frequency components representative of the individual's breathing.

The self-similarity calculation requires no external calibration signal. A measured signal can be used to create recopies (i.e. reference signals) of the measured signal, which in turn can be used to classify subsequent measured signals, as explained in detail below.

In a first sub-step following the optional preliminary sub-step, sets of reference points can be obtained from the signal measured or processed in the optional preliminary sub-step. For this purpose, the signal is sampled, and extrema points can be identified in the signal.

The extrema can be signal maxima and/or minima.

In one variant, reference points can be obtained by applying a time shift to the extrema identified to represent different signal frequencies.

Typically, the time lag is of the order of one or more seconds.

However, any other method can be used to obtain reference points.

Reference point sets can include data corresponding to expected positions for a signal with a certain frequency.

For example, if the process described in FIG. 2 is used to determine the heart rate of a human being, the set of reference points may cover a range between 35 and 110 beats per minute.

Different sets of reference points can be saved for analysis of the measured signal. In one variant, only the identified extrema are saved, and the remaining part of the signal is deleted.

In a second sub-step, a plurality of extrema are identified in the measured signal.

Self-similarity is applied to all or some of the extrema of the measured signal, and in a third sub-step, as a function of a time offset between the measured signal and its recopy, a score is calculated representing the distances between each extremum of the measured signal and the closest extremum of its recopy.

The two-dimensional result of the self-similarity calculation is a score that can be assigned to each reference point, to indicate how close the reference point is to an extremum, so that the highest score is assigned to the reference points with the smallest deviation.

In this way, an self-similarity is an inverse function inverse function of the distances between each extremum of the measured signal and the nearest extremum of its recopy.

The time lag, which is a variable in the self-similarity function, can be bounded, i.e. vary within a specified range.

The comparison of extrema with reference points can include a frequency comparison and/or an amplitude comparison.

In one embodiment, the total score for a set of reference points can be obtained by calculating the sum of the scores assigned to each reference point in a set.

In another embodiment, the total score for a set of reference points can be the maximum of the scores for a set of reference points.

This comparison can be repeated for each set of reference points.

However, any other suitable method can be used to compare the set of reference points that correspond to the extrema identified.

The set of reference points that correspond most closely to the extrema is identified to determine the frequency of the signal to be analyzed.

In order to determine the distance between two neighboring extrema, the time duration of the signal to be analyzed must be long enough to include at least two extrema. For example, if the signal to be analyzed represents cardiac activity with 75 beats per minute, the gap between two maxima is 0.8 seconds, so the sampling time should be at least 1.6 seconds.

Every 1.6 seconds, we obtain a self-similarity curve as a function of time lag. This similarity curve then forms a set of similarity scores, in which each lag is associated with a similarity score (i.e. a similarity score as a function of lag).

In the example in FIG. 3, for sinus rhythm, i.e. normal heart rhythm, the maximum score appears every 0.8 seconds (solid curve in FIG. 3).

In the case of atrial fibrillation, the score fluctuates almost arbitrarily, and no clear signal periodicity can be identified (dotted curves in FIG. 3).

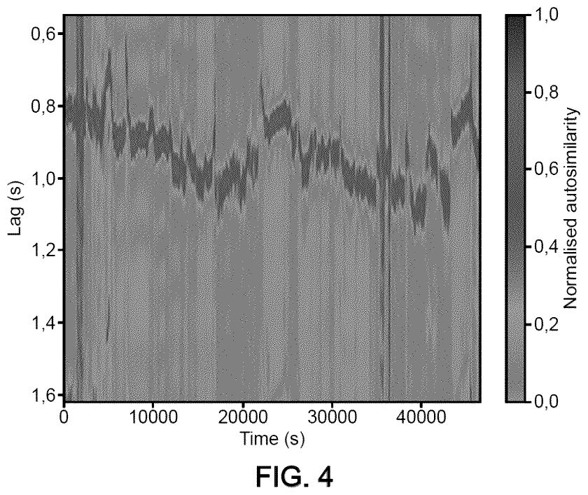

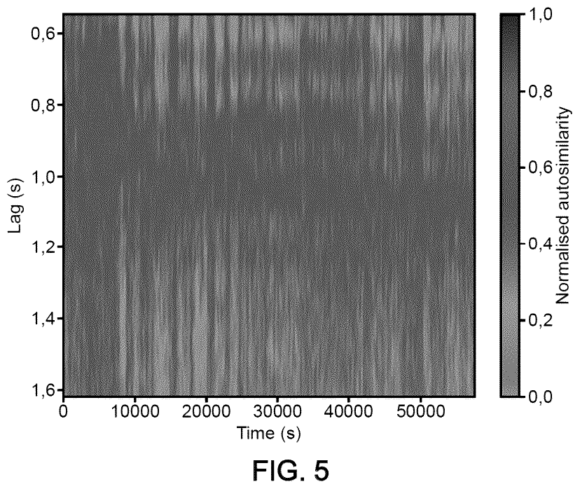

By placing several curves self-similarity curves obtained from signals measured in succession “next to each other”, we obtain a three-dimensional representation of self-similarity as a function of lag (vertical axis) and as a function of time or portions of the signal for which a set of scores has been calculated (horizontal axis), as shown in FIGS. 4 and 5.

That is, a plurality of curves as shown in FIG. 3 are juxtaposed or stacked, so as to form a three-dimensional representation of the temporal evolution of the sets of scores in FIG. 2. This three-dimensional representation thus forms a stack of similarity scores of the representative signal, in which a score is associated with each portion and each offset (score as a function of portion and offset, therefore). The abscissa of FIG. 3 corresponds to the ordinate of FIGS. 4 and 5.

Depending on the individual's type of heart rhythm, the structure of self-similarity differs.

FIG. 4 shows the case of sinus rhythm, and FIG. 5 the case of atrial fibrillation.

Such images typically comprise between 50 and 200 points on each axis, i.e. between 0.0025 and 0.04 megapixels. This relatively small image size minimizes the computing costs involved in analyzing such images.

In the case of sinus rhythm, the maximum of each of the two-dimensional curves forms the dark curve (FIG. 4). More generally, we are interested in the dark areas in the image and the continuity of these dark areas. In other words, in the case of a normal heartbeat, we see a roughly horizontal dark band, not necessarily rectilinear or even continuous. The periodicity of the heartbeat varies mainly between 0.8 seconds and 1.1 seconds.

As explained in the description of FIG. 3, in the case of atrial fibrillation, the self-similarity curve fluctuates almost arbitrarily, and no clear signal periodicity can be identified). In other words, in the case of an abnormal heartbeat, in the image constructed from the juxtaposition of several self-similarity results, it is not possible to distinguish dark areas roughly forming a band (FIG. 5).

Similarity sets and stacking can be calculated with self-similarity, as described above, or with autocorrelation, sliding Fourier transform or wavelet transform. However, self-similarity calculation is particularly suitable for ballistocardiograms.

In another variant, the images can be processed by a “conventional” algorithm and not by a neural network. Such an algorithm may be based, for example, on recognition of the dark area in the images.

The analysis of the images shown in FIGS. 4 and 5 is explained below with the help of FIG. 6.

FIG. 6 shows a two-dimensional convolutional neural network (hereinafter referred to as CNN) for implementing step 204 of the process described in FIG. 2.

This is a special case of an artificial neural network, specially adapted to image analysis, in particular for the detection and analysis of certain patterns in images.

A CNN is an algorithm that enables a processing unit 106 executing this algorithm to implement the principle of learning from experience, i.e. learning by analyzing examples, in this case images according to FIG. 4 or 5, calculated from signals representative of an individual's cardiac activity on a repeated basis.

A large number of images of the same category can be presented to the CNN, for example results of self-similarity calculations, calculated from signals representative of cardiac activity in an adult suffering from atrial fibrillation. The 106 processing unit thus learns to recognize this type of recurring pattern when an unknown image is presented.

The CNN can continue learning as the process is implemented.

Step 204 of the process shown in FIG. 2 can therefore be adapted and optimized on the basis of previously determined image analysis.

The operation of a CNN is based on a large number of processors operating in parallel and organized in layers.

A CNN can comprise an input layer inL, one or more hidden layers hidL, and an output layer outL, arranged one after the other. A posterior layer receives as input the result of a prior layer.

Each layer can comprise several elements.

In the case of the inL input layer, the input element represents images to be analyzed.

An artificial neuron, represented by an arrow, represents a transfer function that transforms the activation of elements in one layer into the activation of elements in the next layer, according to rules that can change, following the principle of automatic learning.

A CNN is used to test functional hypotheses. Each arrow coming from a layer represents a hypothesis being tested.

For example, each image can be analyzed with a sliding filter, and certain patterns can be searched for in the image, such as a signal with a “lag” of between 0.8 seconds and 1.1 seconds.

For an element in a layer, a plurality of hypotheses can be tested. The same hypotheses are tested for each element in a layer.

Each element in a layer receives all the results of a single hypothesis for all the elements in the previous layer.

A first hidden layer hidL receives information outputs from the first layer, i.e. signals processed/analyzed according to a set of assumptions.

If required, depending on the complexity of the algorithm, the output of the second layer can be guided to a second hidden layer. The number of hidden layers, as well as the number of elements in each layer, is not limited, and can be adapted according to the problem to be solved.

By way of example and not limitation, the hidden layers in FIG. 6 comprise four elements each. The elements of a hidden layer can correspond, for example, to the different patterns that can be identified in an image.

The final outL layer produces the system results. In this case, the result could be, for example, “No particularities detected” or “Atrial fibrillation detected: Please contact your doctor”.

In the example shown in FIG. 6, the last outL layer of the CNN can provide three different results: “atrial fibrillation” (AF), “sinus rhythm” (SR) or “error” (err).

In one embodiment, at least some of steps /b/ to /e/ are carried out repeatedly, and the signal characteristic representative of cardiac activity is provided every 1 minute to 5 minutes.

FIG. 7 shows a processing unit 106 adapted to implement the process described in FIG. 2. This processing unit may be part of the device described in FIG. 1.

In this embodiment, the processing unit comprises a memory 110 for storing instructions for implementing at least part of the process, received data, and temporary data for carrying out the various steps and operations of the process as described above.

The processing unit also includes a 111 control circuit. This circuit can be, for example:
a processor capable of interpreting instructions in the form of a computer program, or
an electronic card whose steps and operations of the disclosure process are described in the silicon, or
a programmable electronic chip such as an FPGA (Field-Programmable Gate Array), a SOC (System On Chip) or an ASIC (Application Specific Integrated Circuit).

SOCs or systems-on-a-chip are embedded systems that integrate all the components of an electronic system on a single chip. An ASIC is a specialized electronic circuit that groups together tailor-made functionalities for a given application. ASICs are generally configured at the time of manufacture, and can only be simulated by a user of the processing unit. FPGA-type programmable logic circuits are electronic circuits that can be reconfigured by the user of the processing unit.

The processing unit has an input interface for receiving messages or instructions, and an output interface for communicating with the at least one sensor 107, 108. In the example shown here, the processing unit is integrated into the device shown in FIG. 1.

Depending on the embodiment, the processing unit 106 may be a computer, a computer network, an electronic component, or other apparatus comprising a processor operatively coupled to a memory, as well as, depending on the embodiment chosen, a data storage unit, and other associated hardware elements such as a network interface and a media drive for reading from and writing to a removable storage medium not shown in FIG. 7. The removable storage medium can be, for example, a CD compact disc, a DVD digital video/polyvalent disc, a flash disk, a USB stick, etc.

Depending on the embodiment, memory 110, the data storage unit or the removable storage medium contains instructions which, when executed by control circuit 111, cause this control circuit to perform or control input interfaces, output interfaces, data storage in memory 110 and/or data processing and examples of implementation of the process described in FIG. 2.

The control circuit 111 can be a component implementing the control of the processing unit 106.

In addition, the processing unit 106 can be implemented in software form, in which case it takes the form of a program executable by a processor, or in hardware form, such as an application-specific integrated circuit (ASIC), a system-on-a-chip (SOC), or in the form of a combination of hardware and software elements, for example a software program designed to be loaded and executed on a previously described electronic component such as an FPGA or processor.

The processing unit 106 can also use hybrid architectures, such as CPU+FPGA, GPU (Graphics Processing Unit) or MPPA (Multi-Purpose Processor Array).

The processing unit can control at least some of the device components shown in FIG. 1. In particular, the processing unit can control the pressure sensor 107 and the acoustic sensor 108.

To this end, the processing unit may include storage hardware for storing at least part of the measured signal and/or processed signal and/or characteristic.

The device according to FIG. 1 can be coupled 109 wirelessly, for example via Bluetooth™, Wi-Fi or a cellular network, to one or more devices, such as to a second processing unit such as a “smartphone” 105 or a tablet or a remote server, without excluding direct interfacing of a telemedicine in a remote server.

The present disclosure makes it possible to determine a heart condition of an individual for the provision of a feature.

The present disclosure is not limited to the examples of devices, systems, processes, uses and computer program products described above, by way of example only, but embraces all variants that the person skilled in the art may envisage in the context of the protection sought.

1. A method of determining a characteristic of a cardiac condition of an individual, said method comprising the following steps:
a) obtaining (201) at least one signal representative of a cardiac activity of the individual repeatedly;
c) processing, wherein said processing comprises calculating, for a portion of the representative signal, a set of similarity scores, said set of similarity scores associating similarity score values with respective time shifts;
d) analyzing (204) a temporal evolution of at least part of the at least one processed signal, and
e) providing (205) a characteristic of the signal representative of the cardiac activity based on steps a) to d).

2. (canceled)

3. The method according to claim 1, wherein the at least one signal representative of cardiac activity is a pressure signal and step c) comprises determining a ballistocardiogram.

4. (canceled)

5. The method according to claim 1, wherein the set of similarity scores is calculated by comparing the portion with a plurality of recopies of the portion to which a respective time offset has been applied, a similarity score value being assigned for each time offset.

6. The method according to claim 5, wherein
processing step c) comprises stacking a plurality of sets of similarity scores calculated for a plurality of respective portions, to form a stack of self-similarity scores of the representative signal, said stack thus associating similarity score values of the representative signal with time shifts and portions of the representative signal, and
said stack is analyzed in step d).

7. The method according to claim 1, wherein step c) comprises calculating a self-similarity.

8. The method according to claim 1, in which wherein step d) is implemented at least in part by artificial intelligence, wherein the artificial intelligence comprises an artificial neural network.

9. (canceled)

10. The method according to claim 8, wherein the artificial neural network comprises a two-dimensional convolutional neural network.

11. The method according to claim 1, in wherein at least some of steps c) to e) are carried out repeatedly, and the feature is provided every 1 minute to 5 minutes.

12. The method according to claim 1, further comprising:
b) determining a relevance of at least part of the at least one obtained signal, and wherein a step of notifying the characteristic of the obtained signal is implemented only if step b) determines that the obtained signal is relevant.

13. The method according to claim 12, wherein step b) comprises
analyzing the at least one signal obtained representative of cardiac activity of the individual; and/or
measure and analyze at least one additional signal representative of a movement performed by the individual, a breath taken by the individual or a pressure exerted by the individual.

14. The method according to claim 1, wherein the characteristic of the representative signal relates to the regularity or irregularity of the signal over time.

15. The method according to claim 1, wherein step e) comprises identifying an atrial fibrillation or identifying an absence of a characteristic.

16. The method according to claim 1, wherein at least part of the process is carried out while the individual is asleep.

17. A processing unit for determining a characteristic of a cardiac condition of an individual, the processing unit being configured to implement a method of determining a characteristic of a cardiac condition of an individual according to claim 1.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A device for determining a characteristic of a cardiac condition of an individual, the device comprising at least one sensor configured to measure at least one signal representative of cardiac activity of the individual repeatedly, and the processing unit according to claim 17.

35. The device according to claim 34, configured to determine the individual's cardiac status without being in physical contact with the individual.

36. The device according to claim 34, comprising a second processing unit remote from the device and configured to receive from the first processing unit, after implementation of the process by the first processing uni, data representative of at least part of the at least one measured signal and/or the at least one processed signal and/or the characteristic.

37. The device of claim 36, wherein the second processing unit comprises a smartphone or tablet.

38. A non-transitory computer readable medium comprising instructions for implementing the method according to claim 1 when the instructions are executed by a processor.

39. The method according to claim 12, wherein step b) comprises an automatic learning step, implemented by artificial intelligence.
